(19) United States
(12) Patent Application Publication
(10) Pub. No.: US 2025/0312161 A1
(43) Pub. Date: Oct. 9, 2025

(54) THREE-DIMENSIONAL POROUS STRUCTURES FOR BONE INGROWTH AND METHODS FOR PRODUCING

(71) Applicant: Depuy Ireland Unlimited Company, Ringaskiddy (IE)

(72) Inventors: Edward Patrick Kavanagh, Mallow (IE); David Horne, Leeds (GB); James Oliver Sedicol, Waterford (IE); Philippa Clarkson, Leeds (GB); Fionnán Aodhán McNamara, County Carlow (IE); Enda Leavy, Cork (IE); Karen McKinlay, Everton (GB); João Arranhado Neves, Manor Farm (IE); Charlie Weston, West Yorkshire (GB)

(21) Appl. No.: 19/245,534

(22) Filed: Jun. 23, 2025

Related U.S. Application Data
(62) Division of application No. 17/732,750, filed on Apr. 29, 2022.

(51) Int. Cl. A61F 2/30; A61F 2/34

(52) U.S. Cl. CPC A61F 2/30767; A61F 2/3094; A61F 2/34; A61F 2002/30011; A61F 2002/3092; A61F 2002/3093; A61F 2002/3403; A61F 2002/3408

(57) ABSTRACT

A method of manufacturing an orthopaedic prosthetic component can include identifying a porous three-dimensional Voronoi structure shaped to be implanted in a patient's body. The porous three-dimensional Voronoi structure can include a plurality of struts, a number of pores, a first surface, and a second surface. The plurality of struts can define randomized interconnected organicized cells. Respective groups of struts intersect so as to define a respective plurality of nodes. The method can include the step of modifying modifying at least one of the struts or at least one of the nodes such that the porous three-dimensional structure comprises a lattice structure other than a Voronoi pattern. Instructions can then be generated to fabricate the porous three-dimensional structure.

### CROSS-REFERENCE TO RELATED APPLICATIONS

This is a divisional of U.S. patent application Ser. No. 17/732,750 filed Apr. 29, 2022, the disclosure of which is hereby incorporated by reference as if set forth in its entirety herein.

### TECHNICAL FIELD

The embodiments disclosed herein are generally directed towards porous metal structures and methods for manufacturing them, and, more specifically, to porous metal structures in medical devices that have geometric lattice configurations suited to allow for exact control of porosity and pore size in a porous metal structure.

### BACKGROUND

The embodiments disclosed herein are generally directed towards three-dimensional porous structures for bone ingrowth and methods for producing said structures.

The field of rapid prototyping and additive manufacturing has seen many advances over the years, particularly for rapid prototyping of articles such as prototype parts and mold dies. These advances have reduced fabrication cost and time, while increasing accuracy of the finished product, versus conventional machining processes, such as those where materials (e.g., metal) start as a block of material, and are consequently machined down to the finished product.

However, the main focus of rapid prototyping three-dimensional structures has been on increasing density of rapid prototyped structures. Examples of modern rapid prototyping/additive manufacturing techniques include sheet lamination, adhesion bonding, laser sintering (or selective laser sintering), laser melting (or selective laser melting), photopolymerization, droplet deposition, stereolithography, 3D printing, fused deposition modeling, and 3D plotting. Particularly in the areas of selective laser sintering, selective laser melting and 3D printing, the improvement in the production of high-density parts has made those techniques useful in designing and accurately producing articles such as highly dense metal parts.

In the past few years, some in the additive manufacturing fields have attempted to create solutions that provide the mechanical strength, interconnected channel design, porosity, and pore size in porous structures necessary for application in promoting mammalian cell growth and regeneration. However, the current methods and geometries have limited control over the pore size distribution, which exerts a strong influence on the ingrowth behavior of mammalian cells such as tissue or bone. Moreover, the current methods and geometries often fall short in producing porous structures having unit cell geometries with pore sizes and porosities simultaneously in the range believed to be beneficial for ingrowth while maintaining structural integrity during the manufacturing process (e.g., 3D printing). As a result, current unit cell geometric structures must either have a very large pore size or very low porosity. Furthermore, current methods and geometries generally prevent close correlation between a selected strut length and diameter of a unit cell, within a structure's geometry, and the resulting geometric features desired in the porous structure.

Current methods of manufacturing porous metal materials for bone ingrowth have limited control over the pore size distribution, which exerts a strong influence on the ingrowth behavior of bone. Better simultaneous control of the maximum pore size, minimum pore size, and porosity would enable better bone ingrowth. Additive manufacturing techniques conceptually enable production of lattice structures with perfect control over the geometry but are practically limited to the minimum outer strut diameter that the machine can build, and by the need for any lattice structure to be self-supporting. The minimum strut diameter for current 3D printers is approximately 200-250 microns, which means that many geometric structures must either have a very large pore size or very low porosity.

### SUMMARY

An orthopaedic prosthetic component can include a porous three-dimensional structure shaped to be implanted in a patient's body. The porous three-dimensional structure can include a plurality of struts defining randomized interconnected organicized cells, wherein respective groups of struts intersect so as to define a respective plurality of nodes, The organicized cells can define a number of pores. The porous three-dimensional structure can include a first portion defining a first surface, a second portion defining a second surface spaced from the first surface along a transverse axis, and an intermediate portion between the first surface and the second surface. The first surface can have a first porosity and the intermediate portion can have an intermediate portion porosity that is different from the first porosity.

The second surface can have a second porosity that is different from at least one of the first porosity and the intermediate portion porosity. A ratio of the first porosity to the intermediate portion porosity can be about 1.4:1. The first porosity and the second porosity can each be greater than the intermediate portion porosity. Each strut can include a first end and a second end spaced from the first end along a central axis, each strut having a first cross-sectional shape at a first point along its length in a first plane perpendicular to the central axis, a second cross-sectional shape at a second point along its length in a second plane parallel to the first plane, and the first cross-sectional shape is different from the second cross-sectional shape.

The plurality of organic cells can include a first organic cell having a first seed point within the first organic cell, a second organic cell having a second seed point within the second organic cell, and a third organic cell having a third seed point within the third organic cell. The plurality of struts can include a first strut separating the first organic cell from the second organic cell, the first strut being perpendicular to a straight imaginary line connecting the first seed point to the second seed point, a second strut separating the second organic cell from the third organic cell, the second strut being perpendicular to a straight imaginary line connecting the second seed point to the third seed point, and a third strut separating the third organic cell from the first organic cell, third strut being perpendicular to a straight imaginary line connecting the third seed point to the first seed point.

In a further embodiment, the orthopaedic component can include a mesh coupled to the porous three-dimensional structure at the second surface, the mesh having a mesh porosity that is different than each of the first porosity and the second porosity. Each strut can include a first end and a second end spaced from the first end along a central axis, and less than 1% of the struts have their first end connected to another strut at one of the nodes and their second end is a free hanging end. At least 99% of the struts can have a thickness of about 0.2 millimeters to about 0.4 millimeters. The orthopaedic prosthetic component can have a porosity between about 60% and about 85%. 90 percent of the pores can have a pore size that ranges from 0.5 mm to 2 mm. The orthopaedic prosthetic component can comprise an acetabular cup.

In one embodiment a method of manufacturing an orthopaedic prosthetic component comprises identifying a porous three-dimensional structure defined by a plurality of struts positioned according to a Voronoi pattern of randomized seed points, the struts defining a plurality of interconnected organic cells. The struts can intersect at a plurality of nodes. The method can include modifying at least one of the struts or at least one of the nodes such that the porous three-dimensional structure comprises a lattice structure other than a Voronoi pattern, and fabricating the porous three-dimensional structure by applying an energy source to fusible material.

The modifying step can include organicizing the at least one strut to increase a thickness of a portion of at least one of the struts. The modifying step can include organicizing one of the nodes to increase a thickness of the node. The plurality of struts can cooperate to define a number of pores having window sizes defined as a diameter of a circle positioned in the pores, such that the struts that define the pores are positioned on a tangent line of the circle. The porous three-dimensional structure can have a porosity between about 60% and about 85%.

A method of manufacturing an orthopaedic prosthetic component can include creating a porous three-dimensional structure by causing a computing device to perform the steps of defining a three-dimensional space having an inner boundary and an outer boundary, randomly positioning a plurality of seed points within the three-dimensional space, defining a plurality of cells by a Voronoi structure such that each cell can include one of the seed points, the plurality of cells separated from each other by struts that intersect at a plurality of nodes, modifying at least one of the nodes or the struts such that the porous three-dimensional structure comprises a lattice structure other than a Voronoi structure, and fabricating the porous three-dimensional structure by applying an energy source to fusible material. The fabricating step can include fabricating an acetabular cup.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a perspective elevation view of an orthopaedic prosthetic component;

FIG. 2 is an enlarged elevation view of a portion of the orthopaedic prosthetic component of FIG. 1;

FIG. 3 is a schematic view of seed points for a Voronoi structure;

FIG. 4 is a schematic view of a portion of the seed points of FIG. 3 with connecting lines drawn between adjacent seed points;

FIG. 5 is a schematic view of the seed points of FIG. 4 with bisectors drawn for each connecting line;

FIG. 6 is a schematic view of the seed points and bisectors of FIG. 5 with the bisectors trimmed;

FIG. 7 is a schematic view of the bisectors of FIG. 6 with the seed points removed;

FIG. 8 is a schematic view of a Voronoi structure;

FIG. 9 is an enlarged elevation view of a non-organicized porous structure;

FIG. 10 is an enlarged elevation view of an organicized porous structure;

FIG. 11 is a sectional view of a portion of the prosthetic component of FIG. 1;

FIG. 12 is an isolated, perspective view of the mesh of FIG. 1;

FIG. 13 is an elevation view of a mesh having a grid layout;

FIG. 14 is an elevation view of a mesh having a honeycomb;

FIG. 15 is an elevation view showing the alignment of the struts of FIG. 1 with the mesh of FIG. 12; and

FIG. 16 is a perspective view of the orthopaedic component of FIG. 1 illustrating macrocuts on an outer surface thereof.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the Figs. may show simplified or partial views, and the dimensions of elements in the Figs. may be exaggerated or otherwise not in proportion. In addition, as the terms “on,” “attached to,” “connected to,” “coupled to,” or similar words are used herein, one element (e.g., a material, a layer, a base, etc.) can be “on,” “attached to,” “connected to,” or “coupled to” another element regardless of whether the one element is directly on, attached to, connected to, or coupled to the other element, there are one or more intervening elements between the one element and the other element, or the two elements are integrated as a single piece. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. As used herein, the terms “substantial,” “about,” “approximate,” words of similar import, and derivatives thereof when used with respect to a size, shape, dimension, direction, orientation, or the like include the stated size, shape, dimension, direction, orientation, or the like as well as a range associated with typical manufacturing tolerances, such as plus and minus 2%.

As used herein, “bonded to” or “bonding” denotes an attachment of metal to metal due to a variety of physicochemical mechanisms, including but not limited to: metallic bonding, electrostatic attraction and/or adhesion forces.

Unless otherwise defined, scientific and technical terms used in connection with the present teachings described herein shall have the meanings that are commonly understood by those of ordinary skill in the art.

The present disclosure relates to porous three-dimensional structures and methods for manufacturing them for medical applications. As described in greater detail below, the porous structures promote hard or soft tissue interlocks between prosthetic components implanted in a patient's body and the patient's surrounding hard or soft tissue. For example, when included on an orthopaedic prosthetic component configured to be implanted in a patient's body, the porous three-dimensional structure can be used to provide a porous outer layer of the orthopaedic prosthetic component to form a bone in-growth structure. Alternatively, the porous three-dimensional structure can be used as an implant with the required structural integrity to both fulfill the intended function of the implant and to provide interconnected porosity for tissue interlock (e.g., bone in-growth) with the surrounding tissue. In various embodiments, the types of metals that can be used to form the porous three-dimensional metallic structures can include, but are not limited to, titanium, titanium alloys, stainless steel, cobalt chrome alloys, tantalum, poly-ether-ether-ketone (PEEK), poly-ether-ketone-ketone (PEKK), or niobium.

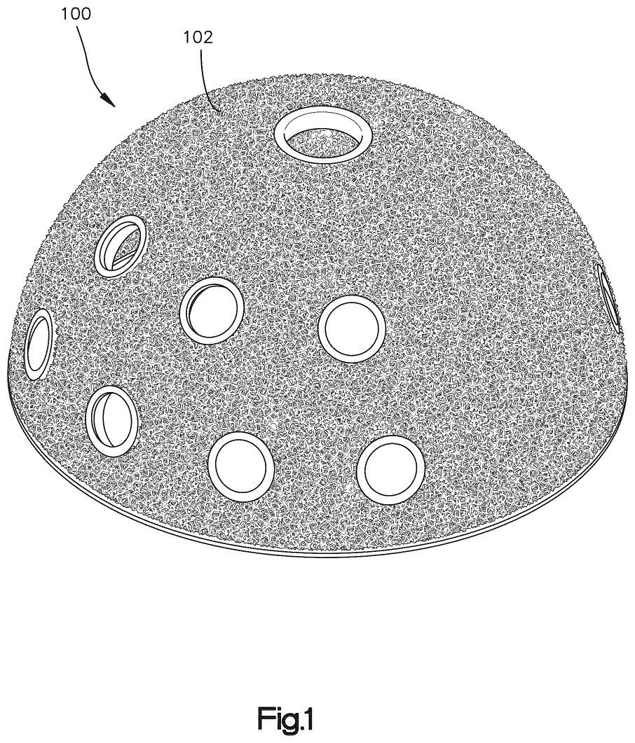

Referring now to FIG. 1, an implantable apparatus such as an orthopaedic implant or prosthetic component 100 is illustrated. The prosthetic component 100 can include a porous three-dimensional structure 102. As described in greater detail below, the porous structure 102 can include a plurality of cells that define pores that permit the ingrowth of bone or soft tissue, thereby promoting fixation of the prosthetic component 100 to a patient's body.

It should be appreciated that the porous structures described herein may be incorporated into various orthopaedic implant designs, including prosthetic components for use in a hip, knee, elbow, ankle, toe, finger, extremities, spine or shoulder arthroplasty surgery. In some embodiments, the orthopaedic implant 100 can be an acetabular cup.

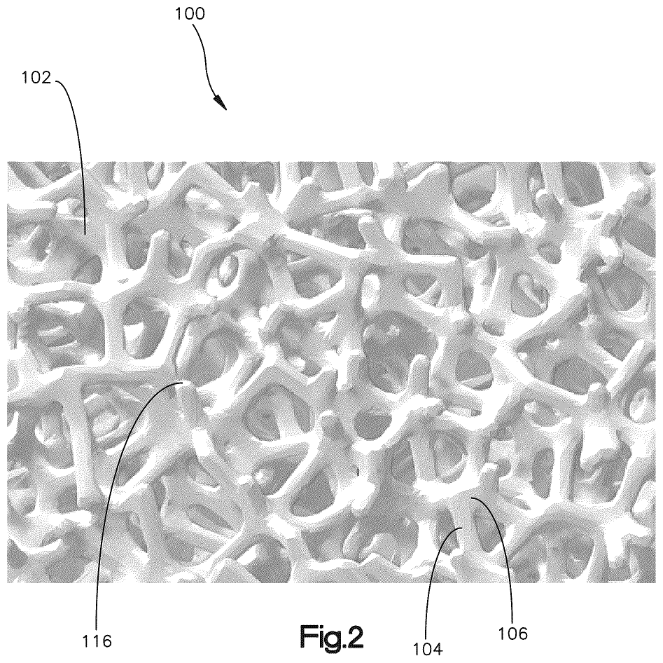

Referring now to FIG. 2, the porous structure 102 of the orthopaedic component 100 can include a plurality of interconnected unit cells. Each unit cell can include a plurality of struts 104 that define a lattice structure. The lattice structure can be a Voronoi structure. The struts 104 can form a three-dimensional perimeter defining the unit cell. Each strut 104 can define a boundary between adjacent unit cells. The struts 104 can include a first end and a second end spaced from the first end along a central axis. At least one of the first and second ends of the struts 104 can be connected to an adjacent strut at a node 106. At least about 99% of the struts 104 can have its first end and its second end connected to an adjacent strut at node 106. Less than about 1% of the struts can be free hanging struts 116. A free hanging strut can have its first end coupled to an adjacent strut at node 106 and its second end being a free end that is not connected to another strut. The free end can have a rounded cylindrical shape. The free hanging struts 116 can extend away from an outer surface of the orthopaedic component by about 175 microns. A free hanging strut can increase friction between the orthopaedic component 100 and an adjacent bone once implanted. The number of free hanging struts may be selected by adjusting the position of the nodes 106 relative to an outer surface of the orthopaedic component 100. Positioning nodes at the outer surface can reduce or eliminate the number of free hanging struts. Spacing the nodes 106 from the outer surface of the orthopaedic component 100 can increase the number of free hanging struts as the struts 104 will extend from the node outwardly to the outer surface without an adjacent node to connect to.

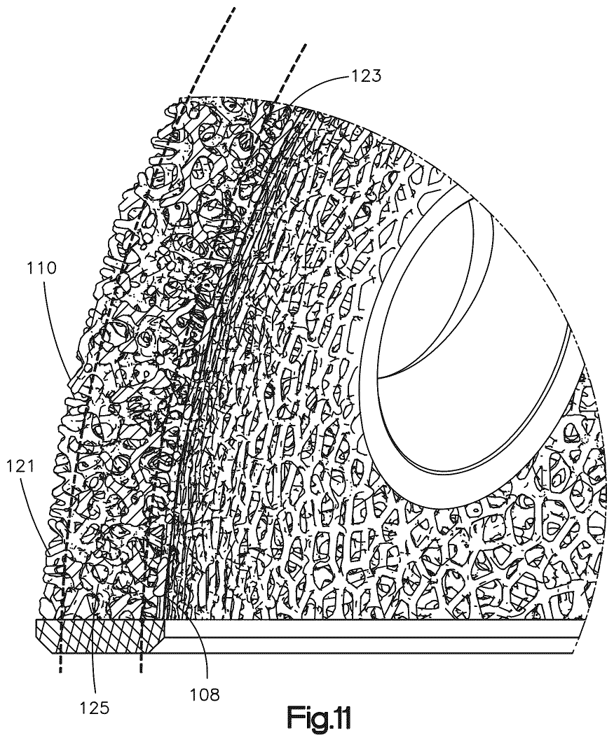

Referring to FIG. 11, the orthopaedic component 100 can include an inner surface 108 and an outer surface 110. The inner surface 108 and outer surface 110 can define the boundary of the orthopaedic component 100. The outer surface 110 can be defined by a select shape. In some embodiments, the outer surface 110 is defined by an oblate hemisphere. The nodes 106 can be recessed from the outer surface 110 such that there are no nodes on the outer surface. The free ends of the free hanging struts can be positioned on the outer surface. The orthopaedic component can have a thickness as measured between the inner surface 108 and the outer surface 110.

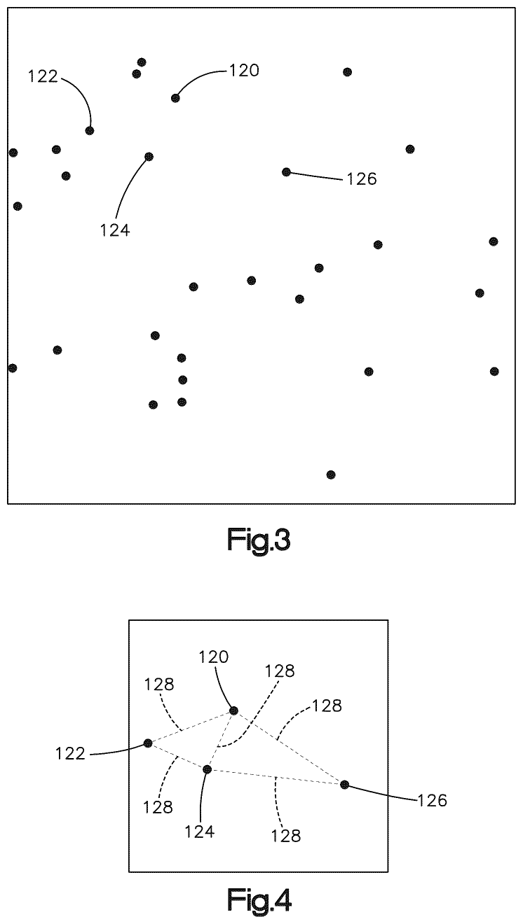

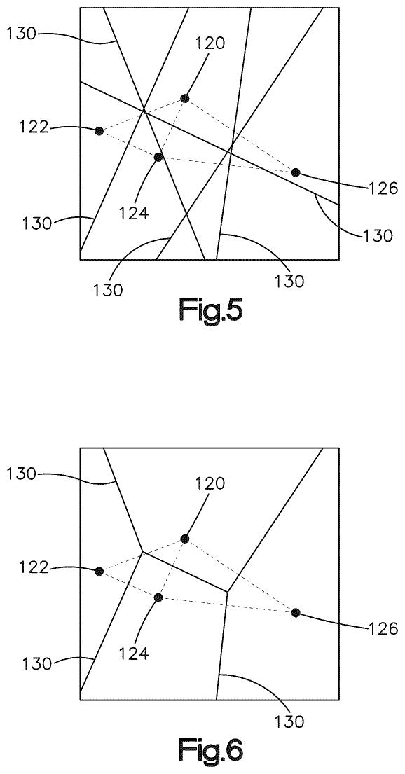

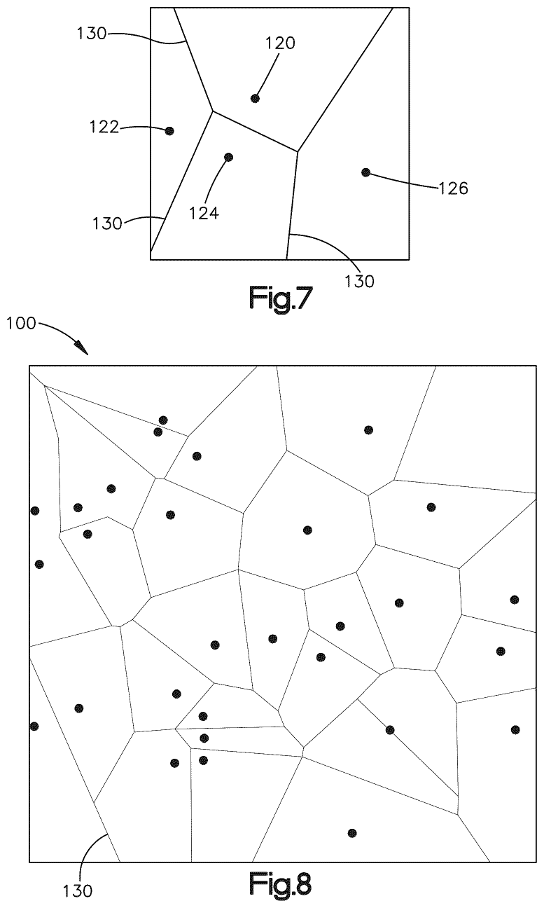

FIGS. 3-8 illustrate one embodiment of creating a Voronoi structure. FIGS. 3-8 show a two-dimensional portion of the orthopaedic component 100 for ease of reference. The same principles can be applied to create the three-dimensional orthopaedic component 100. Referring to FIG. 3, seed points (e.g., 120, 122, 124, 126) can be positioned with the boundary of the orthopaedic component 100. The seed points can be randomly positioned. Randomly positioned seed points can mean that the seed points are not separated from an adjacent seed by a common distance.

FIGS. 4-7 show an isolated view of first seed point 120, second seed point 122, third seed point 124, and fourth seed point 126. Connecting lines 128 are drawn between adjacent seed points (e.g., 120, 122, 124, 126). A bisector 130 is then drawn to bisect each of the connecting lines 128. The bisector 130 can be perpendicular to the connecting line 128. The bisector 130 can intersect the connecting line 128 at a midpoint of the connecting line 128. The bisectors 130 can intersect each other at nodes. The bisectors 130 can define cells. The bisectors 130 can be trimmed such that each cell includes a seed point (e.g., 120, 122, 124, 126) and each seed point is within its own cell. The connecting lines 128 can then be removed such that only the trimmed bisectors 130 remain. The bisectors 130 can be positioned such that any point along the bisector 130 is equidistant from adjacent seed points. The adjacent seed points can be the seed points adjacent to the bisector 130 on opposing sides of the bisector. FIG. 8 illustrates the orthopaedic component 100 of FIG. 3 after the Voronoi design has been applied. The bisectors 130 can define the position of the struts 104 of the orthopaedic component 100.

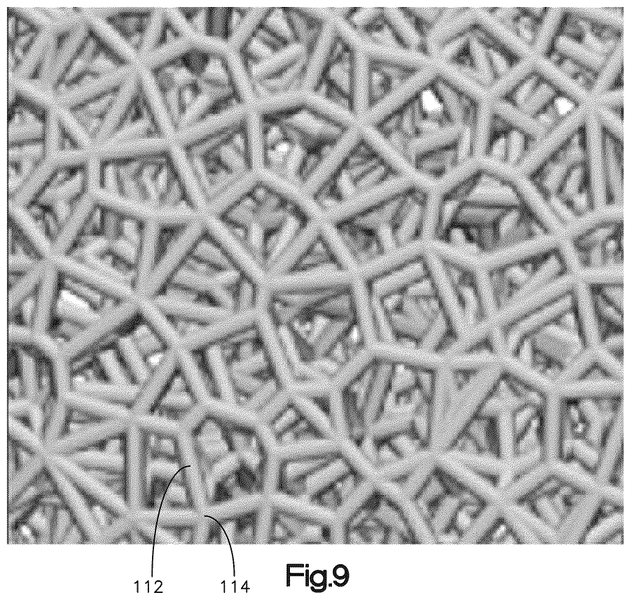

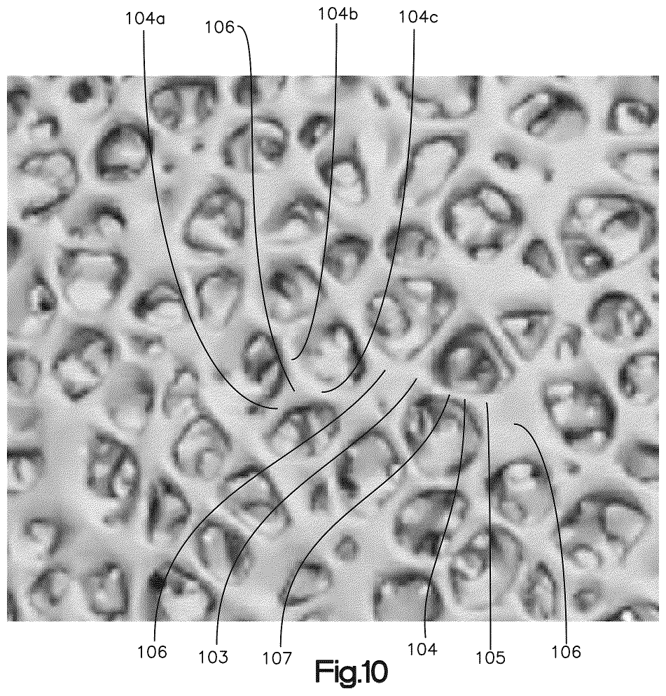

FIG. 9 shows a portion of an orthopaedic component comprising struts 112 of a Voronoi structure having randomly positioned seed points. The struts 112 can be non-organicized. The position of the struts 112 can be defined by the bisecting lines of a Voronoi structure. A non-organicized strut can have a first cross-sectional shape when viewed in a plane perpendicular to the central axis of the strut. The selected first cross-sectional shape can be, for example, a circle, oval, or square. The non-organicized strut 112 can have a uniform cross-sectional shape along its length. Each of the struts 112 can have the same cross-sectional shape. The non-organicized struts 112 can intersect at a plurality of nodes 114. The dimensions of the nodes 114 can be defined by the number of struts 112 that intersect at the node 114 and the angles of the struts 112 relative to each other at the node 114. The spaces between the struts 112 can define pores.

FIG. 10 shows the orthopaedic component of FIG. 9 in a modified state. The modified orthopaedic component 100 can be organicized. An organicized orthopaedic component can more closely resemble cancellous bone than a component with non-organicized struts. Organicizing the orthopaedic component can include adjusting one or more dimensions of the struts 104 or nodes 106. For example, the modified dimension can be a strut shape, thickness, or length. In some embodiments, modifying the strut shape includes modifying the strut shape along only a portion of the length of the strut 104. Alternatively, modifying the strut shape includes modifying the thickness along the length of the strut such that the strut has a uniform, modified thickness, or shape. Organicizing the orthopaedic component can include randomly modifying the shape of portions of the struts.

The organicized strut 104 can have a second cross-sectional shape different from the first cross-sectional shape of the non-organicized strut 112. Although only two different cross-sectional shapes are discussed herein, it should be appreciated that each organicized strut can have more than two cross-sectional shapes at different points along its length (e.g., three, four, five). The second cross-sectional shape can be different at select points along the length of the organicized strut 104. A first portion 103 and second portion 105 of the organicized strut 104 can each be coupled to a node 106. A central portion 107 of the strut 104 can separate the first portion 103 from the second portion 103. The first portion 103 can have a first maximum cross-sectional dimension. The second portion 103 can have a second maximum cross-sectional diameter. The central portion 107 can have a central cross-sectional diameter. The first cross-sectional diameter can be greater than the central cross-sectional diameter. The second cross-sectional diameter can be greater than the central cross-sectional diameter. The first cross-sectional diameter can be equal to the second cross-sectional diameter. The first cross-sectional diameter can different (i.e., less than or greater than) than the second cross-sectional dimeter. At least one of the struts 106 can include a first cross-sectional diameter that is greater than the central cross-sectional diameter. At least one of the struts 106 can include a first cross-sectional diameter that is equal to the central cross-sectional diameter.

An organicized orthopaedic component can include a node 106 having a modified dimension compared to a non-organicized node 114. A non-organicized node can be defined by shape of the struts 104 and the angles of the struts 104 relative to each other at the node 106. An organicized node 106 can include a fillet at the intersection of adjacent struts 104. An organicized node 106 can include a first strut 104a, a second strut 104b, and a third strut 104c. The fillet between the first strut 104a and the second strut 104b can be different than the fillet between the second strut 104b and the third strut 104c.

Organicizing the orthopaedic component can include modifying the lattice structure defined by the struts 104 and nodes 106 such that the lattice is no longer a Voronoi structure. For example, a point that lies on one of the organicized struts may not be equidistant to the adjacent seed points. Organicizing the orthopaedic component 100 can include increasing the thickness or shape of a node 106 or strut 104 such that a pore defined by the struts 104 is eliminated and is instead presented as a solid surface.

The orthopaedic component 100 can have a porosity of between about 70% and about 85%. As discussed above, the term “about” refers to a range associated with typical manufacturing tolerances. In that way, a porosity of “about 70%” may be porosity of 70% plus or minus a typical manufacturing tolerance such as, for example, 2% (i.e., a range of 68% to 72%). In other embodiments, the porosity of the porous three-dimensional structure is between about 20% and about 95%. In other embodiments, the porosity is in a range of between about 35% and about 85%. Geometrically, the porosity of the organic cell structure is dependent on the ratio of the strut length to the strut diameter. Organicizing the orthopaedic component 100 can include modifying the porosity of the orthopaedic component 100. An organicized component can have a lower porosity than a non-organicized structure when each of the organicized component and non-organicized component are based on the same Voronoi structure. The porosity at the inner surface 108 can be less than the porosity at the outer surface 110. The porosity of the outer surface 110 can be selected to allow a substance (e.g., bone cement) to at least partially enter the porous structure 102. The porosity of the inner surface 108 can be selected to prevent the substance from flowing through the inner surface 108.

Referring to FIG. 11, a cross-section of a portion of the orthopaedic component 100 is shown. The orthopaedic component 100 can include a first portion 121 adjacent the inner surface 108, a second portion 123 adjacent the outer surface 110, and an intermediate portion 125 between the first portion 121 and the second portion 123. The first portion 121 can have a first porosity. The second portion 123 can have a second porosity. The intermediate portion 125 can have an intermediate porosity. The first porosity can be greater than the intermediate porosity. The second porosity can be greater than the intermediate porosity. The first porosity can be about 3% to about 30% greater than or less than the second porosity. The first porosity and the second porosity can each be greater than the intermediate porosity. The first porosity can be about 70% to about 95%, about 70% to about 75%, about 75% to about 80%, about 80% to about 85%, about 85% to about 90%, or about 90% to about 95%. The second porosity can be about 60% to about 65%, about 65% to about 70%, about 70% to about 75%, about 75% to about 80%, about 80% to about 85%, or about 60% t0 about 85%. The intermediate porosity can be about 60% to about 65%, about 65% to about 70%, about 70% to about 75%, about 75% to about 80%, about 80% to about 85%, or about 60% to about 85%. The orthopaedic component 100 can have a thickness that extends from the inner surface 108 to the outer surface 110. The first portion 121 can be about 5-25% of the orthopaedic component thickness. The second portion 123 can be about 5-25% of the orthopaedic component thickness. The intermediate portion 125 can be about 50-80% of the orthopaedic component thickness.

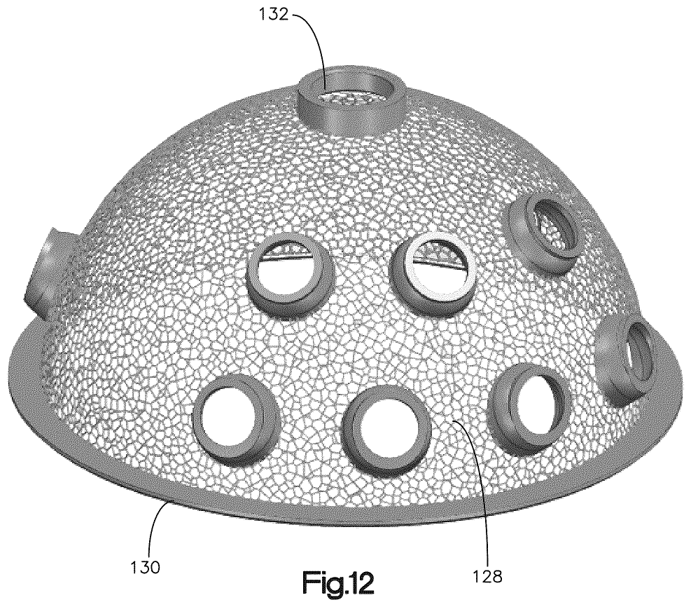

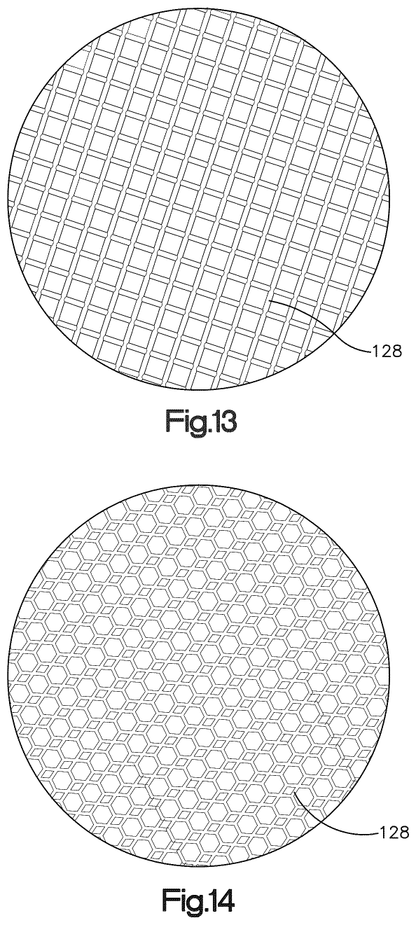

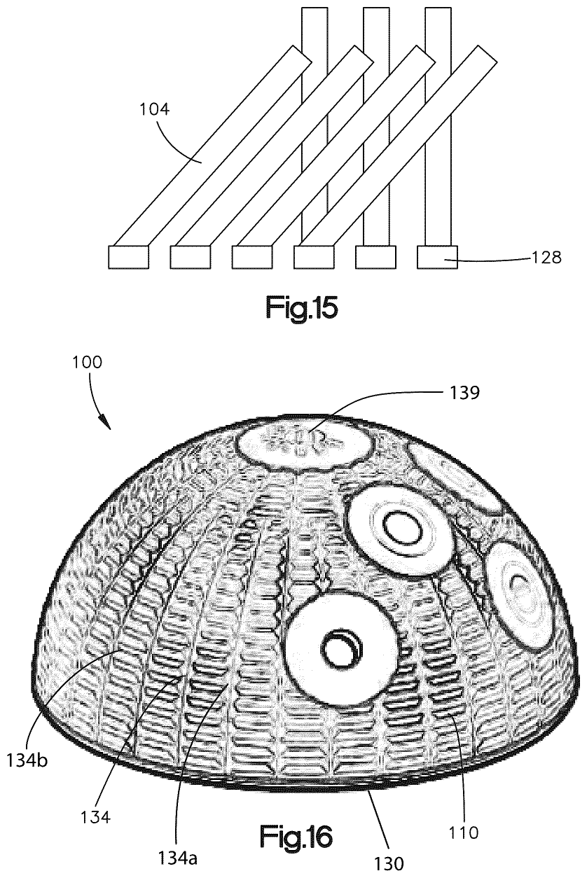

The orthopaedic component 100 can include a mesh. One mesh that can be incorporated into the orthopaedic component is described in U.S. patent application Ser. No. 17/117,166 filed Dec. 10, 2020, and entitled “Acetabular Implant with Predetermined Modulus and Method of Manufacturing Same”, the disclosure of which is hereby incorporated by reference herein. Referring now to FIGS. 12-14, a mesh 128 can define the inner surface 108 of the orthopaedic component 100. The material forming the mesh 128 can be titanium, titanium alloys, stainless steel, cobalt chrome alloys, tantalum, or niobium. The mesh 128 and the porous structure 102 can be the manufactured from the same material. The mesh 128 can provide a scaffold such that the porous structure 102 can be created by additive manufacturing onto the mesh 128, as explained below. The mesh 128 can include a lattice defining a Voronoi pattern (FIG. 12). Alternatively, the mesh 128 can include a lattice defining a square pattern (FIG. 13) or a honeycomb pattern (FIG. 14). The mesh 128 can include a plurality of struts that define the lattice structure. The struts 104 of the porous structure 102 can be aligned with the struts of the mesh 128 (FIG. 15) to reduce or eliminate any free hanging struts on the inner surface 108. The orthopaedic component 100 can include free hanging struts 116 on the outer surface 110 of the porous structure while the inner surface 108 may not include free hanging struts. The mesh 128 can form about 1% to about 10%, about 2% to about 8%, or about 3% to about 5% of the orthopaedic component thickness. The mesh 128 can form about 1% to about 10%, about 2% to about 8%, or about 3% to about 5% of the second portion 123. The mesh 128 can have a porosity that is different from the second porosity. The mesh 128 can have a porosity of about 30% to about 40%, about 40% to about 50%, about 50% to about 60%, about 60% to about 70%, or about 30% to about 70%. The inner portion of the porous structure 102 adjacent the mesh 128 can have a porosity that is different from the mesh porosity.

A rim 130 can be coupled to the mesh 128. The rim 130 can be titanium, titanium alloys, stainless steel, cobalt chrome alloys, tantalum, or niobium. The rim 130 can present a solid surface on which the porous structure 102 is created. The rim 128 can include a width generally equal to the thickness of the orthopaedic component 100. The orthopaedic component 100 can include rings 132. The rings 132 can define an opening adapted to receive a fastener such that the orthopaedic component 100 can be fixed to a bone by the fastener. The rings 132 can extend from the inner surface 108 to the outer surface 110.

Referring to FIG. 16, an outer surface of the orthopaedic component 100 can be textured to increase friction between the orthopaedic component 100 and a bone. The orthopaedic component 100 can include macrocuts 134 on the outer surface 110. The macrocuts 134 are illustrated without the porous structure in FIG. 16 but it should be appreciated that the macrocuts 134 can be formed on the porous structure. Some of the macrocuts 134 can extend in a longitudinal direction. Some of the macrocuts 134 can extend laterally. The longitudinal cuts 134a can extend from the rim 130 toward an apex 139 of the orthopaedic component 100. In some embodiments, the longitudinal cuts 134a extend from the rim 130 to the apex 139. The longitudinal cuts 134a can have a depth as measured from the outer surface 110 toward the inner surface 108. The depth can be about 0.1 mm to about 2 mm, about 0.25 mm to about 1 mm, or about 0.4 mm to about 0.5 mm. The longitudinal cuts 134a can be laterally spaced about the perimeter of the orthopaedic component. The longitudinal cuts 134a can be laterally separated from each other by about 5 degrees to about 30 degrees, about 10 degrees to about 25 degrees, about 15 degrees to about 20 degrees, about 10 degrees, about 15 degrees, about 20 degrees, or about 25 degrees. The porosity of the orthopaedic component at the apex 139 can be different than the porosity adjacent the rim 130.

The lateral cuts 134b can be aligned in a plurality of rows longitudinally spaced from each other. An upper edge of each lateral cut 134b in a row of lateral cuts can be longitudinally aligned. Each row of lateral cuts 134b can be spaced from each other about 1 mm to about 10 mm, about 2 mm to about 8 mm, about 3 mm to about 5 mm, about 2 mm, about 3 mm, about 4 mm, or about 5 mm.

A method is provided for designing the organic cells described herein, having a porous organic three-dimensional structure configured to encourage bone or tissue ingrowth when implanted in a human body. The method can include the step of generating an organic cell design in the manner described above by applying the Voronoi design. In one example, the applying the Voronoi design step can be performed using an NX software package commercially available from Siemens having a place of business in Plano, Texas. The method can include organicizing the lattice structure.

It is recognized that manufacturing tolerances can result in different strut shapes. However, different strut shapes as described herein refers to different shapes outside of manufacturing tolerances.

Once the organic cell design has been produced, manufacturing instructions can be generated to fabricate the porous three-dimensional structure including a plurality of interconnected organic cells. The porous three-dimensional structure can be manufactured on-site. Alternatively, the manufacturing instructions can be sent to a third-party manufacturer to fabricate the porous three-dimensional structure.

The porous three-dimensional metallic structures disclosed above can be made using a variety of different additive manufacturing techniques. For instance, in accordance with various embodiments, a method for producing the porous three-dimensional structure 100 comprises depositing and scanning successive layers of metal powders with a beam. The beam (or scanning beam) can be an electron beam. The beam (or scanning beam) can be a laser beam.

Regarding the various methods described herein, the metal powders can be sintered to form the porous three-dimensional structure. Alternatively, the metal powders can be melted to form the porous three-dimensional structure. The successive layers of metal powders can be deposited onto a rim 130. In various embodiments, the types of metal powders that can be used include, but are not limited to, titanium, titanium alloys, stainless steel, cobalt chrome alloys, tantalum, or niobium powders.

In accordance with various embodiments, a method for producing a porous three-dimensional structure is provided, the method comprising introducing a continuous feed of metal wire onto a base surface and applying a beam at a predetermined power setting to an area where the metal wire contacts the base surface to form a porous three-dimensional structure comprising a plurality of unit cells and having predetermined geometric properties. The beam (or scanning beam) can be an electron beam. The beam (or scanning beam) can be a laser beam. In various embodiments, the types of metal wire that can be used include, but are not limited to, titanium, titanium alloys, stainless steel, cobalt chrome alloys, tantalum, or niobium wire.

In accordance with various embodiments, a method for producing a porous three-dimensional structure is provided, the method comprising introducing a continuous feed of a polymer material embedded with metal elements onto a base surface. The method can further comprise applying heat to an area where the polymer material contacts the base surface to form a porous three-dimensional structure comprising a plurality of organic cells and having predetermined geometric properties. The metal elements can be a metal powder. In various embodiments, the continuous feed of the polymer material can be supplied through a heated nozzle thus eliminating the need to apply heat to the area where the polymer material contacts the base surface to form the porous three-dimensional structure. In various embodiments, the types of metal elements that can be used to embed the polymer material can include, but are not limited to, titanium, titanium alloys, stainless steel, cobalt chrome alloys, tantalum, or niobium.

The method can further comprise scanning the porous three-dimensional structure with a beam to burn off the polymer material. The beam (or scanning beam) can be an electron beam. The beam (or scanning beam) can be a laser beam.

In accordance with various embodiments, a method for producing a porous three-dimensional structure is provided, the method comprising introducing a metal slurry through a nozzle onto a base surface to form a porous three-dimensional structure comprising a plurality of unit cells and having predetermined geometric properties. In various embodiments, the nozzle is heated at a temperature required to bond metallic elements of the metal slurry to the base surface. In various embodiments, the metal slurry is an aqueous suspension containing metal particles along with one or more additives (liquid or solid) to improve the performance of the manufacturing process or the porous three-dimensional structure. In various embodiments, the metal slurry is an organic solvent suspension containing metal particles along with one or more additives (liquid or solid) to improve the performance of the manufacturing process or the porous three-dimensional structure. In various embodiments, the types of metal particles that can be utilized in the metal slurry include, but are not limited to, titanium, titanium alloys, stainless steel, cobalt chrome alloys, tantalum, or niobium particles.

In accordance with various embodiments, a method for producing a porous three-dimensional structure is provided, the method comprising introducing successive layers of molten metal onto a base surface to form a porous three-dimensional structure comprising a plurality of organic cells and having predetermined geometric properties. Further, the molten metal can be introduced as a continuous stream onto the base surface. The molten metal can also be introduced as a stream of discrete molten metal droplets onto the base surface. In various embodiments, the types of molten metals that can be used include, but are not limited to, titanium, titanium alloys, stainless steel, cobalt chrome alloys, tantalum, or niobium.

In accordance with various embodiments, a method for producing a porous three-dimensional structure is provided, the method comprising applying and photoactivating successive layers of photosensitive polymer embedded with metal elements onto a base surface to form a porous three-dimensional structure comprising a plurality of organic cells and having predetermined geometric properties. In various embodiments, the types of metal elements that can be used to embed the polymer material can include, but are not limited to, titanium, titanium alloys, stainless steel, cobalt chrome alloys, tantalum, or niobium.

In accordance with various embodiments, a method for producing a porous three-dimensional structure is provided, the method comprising depositing and binding successive layers of metal powders with a binder material to form a porous three-dimensional structure comprising a plurality of organic cells and having predetermined geometric properties. In various embodiments, the types of metal powders that can be used include, but are not limited to, titanium, titanium alloys, stainless steel, cobalt chrome alloys, tantalum, or niobium powders.

The method can further include sintering the bound metal powder with a beam. The beam (or scanning beam) can be an electron beam. The beam (or scanning beam) can be a laser beam.

In accordance with various embodiments, a method for producing a porous three-dimensional structure is provided, the method comprising depositing droplets of a metal material onto a base surface and applying heat to an area where the metal material contacts the base surface to form a porous three-dimensional structure comprising a plurality of unit cells and having predetermined geometric properties. The beam (or scanning beam) can be an electron beam. The beam (or scanning beam) can be a laser beam. In various embodiments, the types of metal materials that can be used include, but are not limited to, titanium, titanium alloys, stainless steel, cobalt chrome alloys, tantalum, or niobium.

The deposited droplets of metal material can be a metal slurry embedded with metallic elements. The metal material can be a metal powder.

Although specific embodiments and applications of the same have been described in this specification, these embodiments and applications are exemplary only, and many variations are possible.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

1. A method of manufacturing an orthopaedic prosthetic component comprising:
identifying a porous three-dimensional structure defined by a plurality of struts positioned according to a Voronoi pattern of randomized seed points, the struts defining a plurality of interconnected organic cells, the struts intersecting at a plurality of nodes;
modifying at least one of the struts or at least one of the nodes such that the porous three-dimensional structure comprises a lattice structure other than a Voronoi pattern; and
after the modifying step, generating instructions to fabricate the porous three-dimensional structure, such that the orthopaedic prosthetic component is organicized.

2. The method of claim 1, further comprising fabricating the porous three-dimensional structure by applying an energy source to fusible material, based on the generated instructions, wherein the struts are metallic.

3. The method of claim 1, wherein the modifying step includes organicizing the at least one strut to increase a thickness of a portion of at least one of the struts.

4. The method of claim 1, wherein after the modifying step, the plurality of struts cooperate to define a number of pores having window sizes defined as a diameter of a circle positioned in the pores, such that the struts that define the pores are positioned on a tangent line of the circle.

5. The method of claim 1, wherein after the modifying step, the porous three-dimensional structure has a porosity between about 60% and about 85%.

6. The method of claim 1, wherein after the modifying step, each strut of the plurality of struts includes a first end and a second end spaced from the first end along a central axis, each strut having a first cross-sectional shape at a first point along its length in a first plane perpendicular to the central axis, a second cross-sectional shape at a second point along its length in a second plane parallel to the first plane, and the first cross-sectional shape is different from the second cross-sectional shape.

7. The method of claim 1, wherein after the modifying step, each strut of the plurality of struts includes a first end and a second end spaced from the first end along a central axis, and less than 1% of the struts have their first end connected to another strut at one of the nodes and their second end is a free hanging end.

8. The method of claim 1, further comprising, after the modifying step, adjusting a position of the nodes relative to an outer surface of the orthopaedic prosthetic component 100 so as to reduce or eliminate free hanging struts.

9. The method of claim 1, wherein after the modifying step, at least 99% of the struts have a thickness of about 0.2 millimeters to about 0.4 millimeters.

10. The method of claim 1, wherein after the modifying step, 90 percent of the pores have a pore size that ranges from 0.5 mm to 2 mm.

11. The method of claim 1, wherein the struts of the porous three-dimensional structure of the instructions are metallic.

12. The method of claim 11, wherein after the modifying step, a first strut and a second strut of each node intersect so as to define a respective first fillet, and the second strut and a third strut of each node intersect to define a respective second fillet.

13. The method of claim 1, further comprising prior to the modifying step, creating a plurality of bisectors that bisect and extend perpendicular to respective connecting lines that extend between respective adjacent ones of the seed points, wherein intersecting bisectors of the plurality of bisectors intersect each other at the nodes.

14. The method of claim 13, wherein the struts are identified by respective bisectors of the plurality of bisectors, the method further comprising, before the modifying step, trimming the bisectors such that 1) each of the interconnected organic cells includes a respective one of the plurality of seed points, and 2) each seed point of the plurality of seed points is within its own interconnected organic cell.

15. A method of manufacturing an orthopaedic prosthetic component comprising:
creating a porous three-dimensional structure by causing a computing device to perform the steps of:
defining a three-dimensional space having an inner boundary and an outer boundary;
randomly positioning a plurality of seed points within the three-dimensional space;
defining a plurality of cells by a Voronoi structure such that each cell includes one of the seed points, the plurality of cells separated from each other by struts that intersect at a plurality of nodes;
modifying at least one of the nodes or the struts such that the porous three-dimensional structure comprises a lattice structure other than a Voronoi structure; and
after the modifying step, generating instructions to fabricate the porous three-dimensional structure, such that the orthopaedic prosthetic component is organicized.

16. The method of claim 15, further comprising fabricating the porous three-dimensional structure by applying an energy source to fusible material, such that the struts are metallic.

17. The method of claim 15, wherein the fabricating the porous three-dimensional structure comprises fabricating an acetabular cup.

18. The method of claim 15, wherein the struts of the porous three-dimensional structure of the instructions are metallic.

19. The method of claim 15, further comprising prior to the modifying step, creating a plurality of bisectors that bisect and extend perpendicular to respective connecting lines that extend between respective adjacent ones of the seed points, wherein the nodes are defined by intersecting bisectors of the plurality of bisectors.

20. The method of claim 15, wherein the struts are identified by respective bisectors of the plurality of bisectors, the method further comprising, before the modifying step, trimming the bisectors such that 1) each of the interconnected organic cells includes a respective one of the plurality of seed points, and 2) each seed point of the plurality of seed points is within its own interconnected organic cell.
